(19) United States
(12) Patent Application Publication
(10) Pub. No.: US 2020/0138458 A1
(43) Pub. Date: May 7, 2020

(54) CATHETER DEVICE

(71) Applicant: Chang Gung Memorial Hospital, Linkou, Taoyuan City (TW)

(72) Inventors: HSIN-FU LEE, Taoyuan City (TW); PAO-HSIEN CHU, Taoyuan City (TW)

(21) Appl. No.: 16/182,729

(22) Filed: Nov. 7, 2018

(51) Int. Cl. A61B 17/22; A61B 6/00; A61M 25/06; A61M 25/00

(52) U.S. Cl. CPC A61B 17/22; A61B 6/481; A61B 6/504; A61B 2017/22038; A61M 25/0023; A61B 2017/22001; A61M 25/0662

(57) ABSTRACT

The present invention provides a catheter device. This novel catheter device comprises: a catheter, a catheter sheath, a guide wire, a tapered head, a helical structure, and a rotary member. When the catheter is inserted through a blood vessel and stopped by a plaque, it is able to operate the rotary member to rotate the catheter, such that a blockage region of the plaque for the catheter is formed with a through hole therein. Therefore, the guide wire can pass through the through hole of the blockage region, and then the front-end of the catheter can subsequently pass through the blockage region with the forward movement of the guide wire. Consequently, a balloon angioplasty or a stent implantation can easily be conducted in the blockage region of the artery.

### BACKGROUND OF THE INVENTION

### 1. Field of the Invention

The present invention relates to technology filed of catheters, and more particularly to a catheter device for percutaneous coronary intervention (PCI) and endovascular intervention.

### 2. Description of the Prior Art

Cardiovascular disease is one of the top five leading causes of death in Taiwan for a long time. With westernization of diet and growth of aging populations, the cardiovascular diseases are found to get gradually-growing mortality and prevalence rate of year-after-year, and have a tendency of getting increasingly young. Clinical analysis has reported that most individuals suffer from coronary artery disease (CAD) and/or peripheral artery disease (PAD), which are two different types of cardiovascular disease. Cardiologists should know that pathogenesis of these two artery diseases are mainly resulted from that fatty deposits build up along the inner linings of the artery walls, wherein the continuously-deposited fat eventually form calcified plaque to partially or completely occlude the blood vessels. As a result, arterial plaque reduces blood flow or blocks that in some instances, such that heart attack, stroke, or heart failure is therefore induced. Seriously speaking, any one of the aforesaid three heart diseases may induce sudden cardiac death to the patients. On the other hand, PAD encompasses chronic limb ischemia, which progresses into critical limb ischemia (CLI) leading to the distal limb at risk of amputation, and acute limb ischemia, with a rapid loss of blood flow damaging tissue within hours.

Medicine, bypass surgery, percutaneous coronary intervention (PCI) and endovascular intervention are four different treatments for CAD and PAD. In PCI treatment, a catheter is inserted through an artery of the patient to introduce the distal end of the catheter to the obstructive lesion of the artery. Besides, a balloon is disposed on the catheter. Afterwards, an inflation component inflates the balloon with a gas, causing the obstructive lesion of the artery to become more open. PCI treatment also involves the introduction into the artery of a stent, which generally has a metal tubular meshwork structure and is mounted on the inflatable balloon at the distal end of the catheter, through the obstructive lesion of the artery, and the introduction of the balloon and the stent to the obstructive lesion of the artery. Subsequently, the balloon filled with gas expands the stent so as to make the stent to undergo a plastic deformation, thereby installing the stent within the walls of the artery. After the balloon is deflated and pulled back out of the artery, the stent is left behind in place so as to keep the blockage open by continuously supporting the interior walls of the artery with radial force, and the blood flow of the artery return to normal. However, although a guide wire already passed through the blockage region, clinical evidence indicates that, the catheter or equipment (e.g. balloon catheter) cannot pass through the blockage region due to the severe calcification, causing the intervention be unable to successfully completed.

In view of that, some improvements have been made by medical device manufacturers. Please refer to FIG. 1, which illustrates a stereo structural view of a conventionally-used catheter device. As shown in FIG. 1, the catheter device 1′ comprises: a catheter 10′ and a helical structure 11′ formed on the catheter 10′, wherein a drill 12′ is disposed on the front-end of the catheter 10′. By such structural design, when the catheter 10′ is inserted through a blood vessel, it is able to rotate the helical structure 11′ so as to make the drill 12′ pass through the blockage plaque by drilling Therefore, the guide wire 10′ can pass through the through hole of the blockage region, and then the front-end of the catheter 10′ can subsequently pass through the blockage region with the forward movement of the guide wire. Furthermore, after replacing the catheter device 1′ by a balloon catheter, a balloon disposed on the front end of the balloon catheter can reach the blockage region by following the guide wire's move. Consequently, a balloon angioplasty is conducted to make the blockage region of the artery become wider, such that the blood in the artery is able to flow through the blockage region normally.

However, it is further found that in clinical surgery, the catheter 10′ is hard to drill through the plaque of the blockage region because the cylinder-shaped drill 12′ and located on the front-end of the catheter 10′. On the other hand, even if the drill 12′ can successfully drill into the plaque of the blockage region, the helical device may also get stuck in the blockage region. At this time, rupture of blood vessel may occur if forcibly pulling the catheter 10′.

Through above descriptions, it is known that the conventional catheter device 1′ shows many drawbacks and shortcomings in practical use. Accordingly, inventors of the present application have made great efforts to make inventive research thereon and eventually provided a catheter structure.

### SUMMARY OF THE INVENTION

The primary objective of the present invention is to disclose a catheter structure, comprising: a catheter, a guide wire, a catheter sheath, a c tapered head, a rotary member, and a helical structure. When the catheter is inserted through a blood vessel and stopped by a plaque, it is able to operate the rotary member to rotate the catheter, such that a blockage region of the plaque for the catheter is formed with a through hole therein. Therefore, the guide wire can pass through the through hole of the blockage region, and then the front-end of the catheter can subsequently pass through the blockage region with the forward movement of the guide wire. Consequently, a balloon angioplasty or a stent implantation can easily be conducted in the blockage region of the artery.

For achieving the primary objective of the present invention, the inventor of the present invention provides an embodiment for the catheter structure, comprising:
a catheter, having a front-end portion and a tail-end portion;
a catheter sheath, being sleeved on the catheter and slidable;
a tapered head, being connected to the front-end portion;
a guide wire, being disposed in the catheter, and one end of the guide wire extending out of the tapered head from the internal of the catheter;
a rotary member, being connected to the tail-end portion, and the other end of the guide wire passing though the rotary member; and
a helical structure, being formed on one side of the front-end portion.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a stereo structural view of a conventionally-used catheter device;

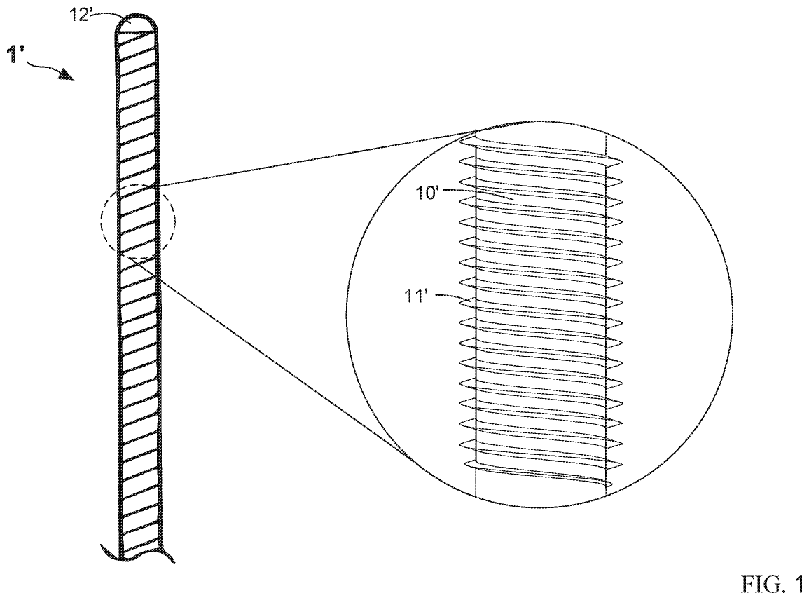

FIG. 2 shows a first stereo view of a catheter device according to the present invention;

FIG. 3 shows an assembly framework view of the catheter device;

FIG. 4 shows a second stereo view of the catheter device;

FIG. 5 shows a first schematic view of the catheter device;

FIG. 6 shows a second schematic view of the catheter device;

FIG. 7 shows a third schematic view of the catheter device;

FIG. 8 shows a fourth schematic view of the catheter device;

FIG. 9 shows a fifth schematic view of the catheter device;

FIG. 10 shows a sixth schematic view of the catheter device; and

FIG. 11 shows a seventh schematic view of the catheter device.

### DETAILED DESCRIPTION OF THE PREFERRED EMBODIMENTS

To more clearly describe a catheter structure, embodiments of the present invention will be described in detail with reference to the attached drawings hereinafter.

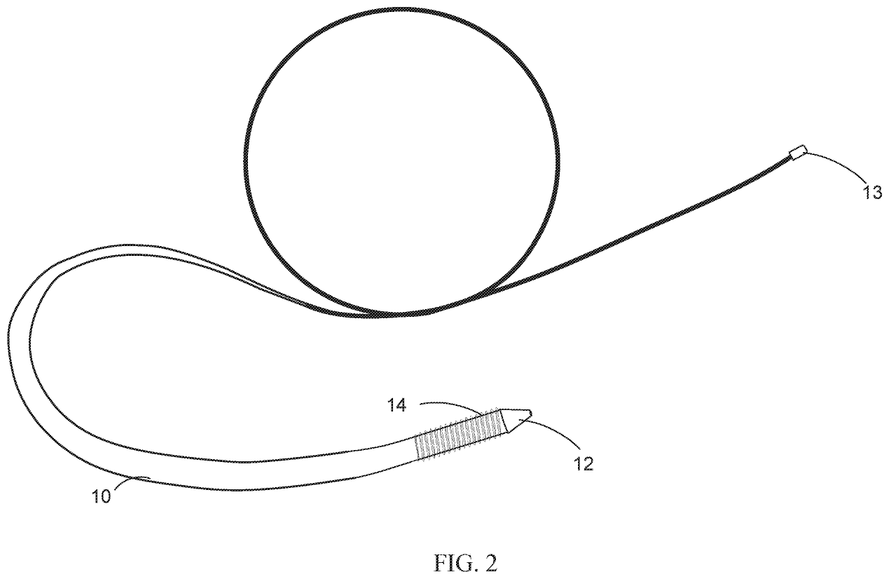

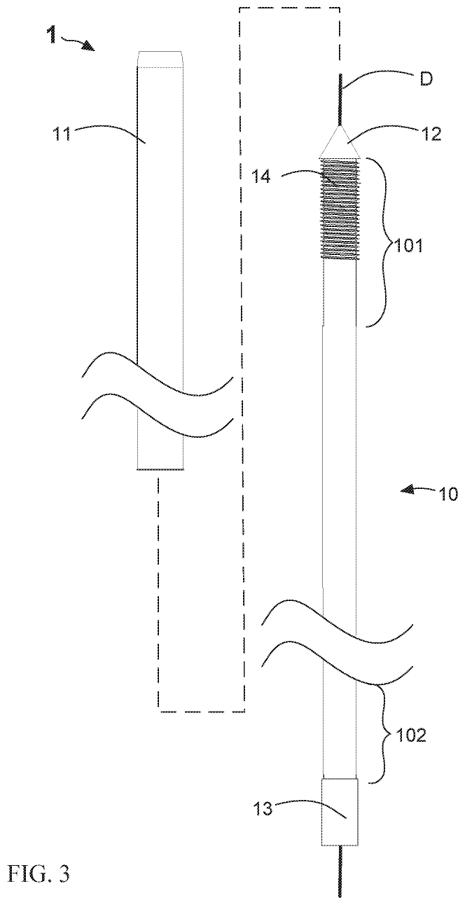

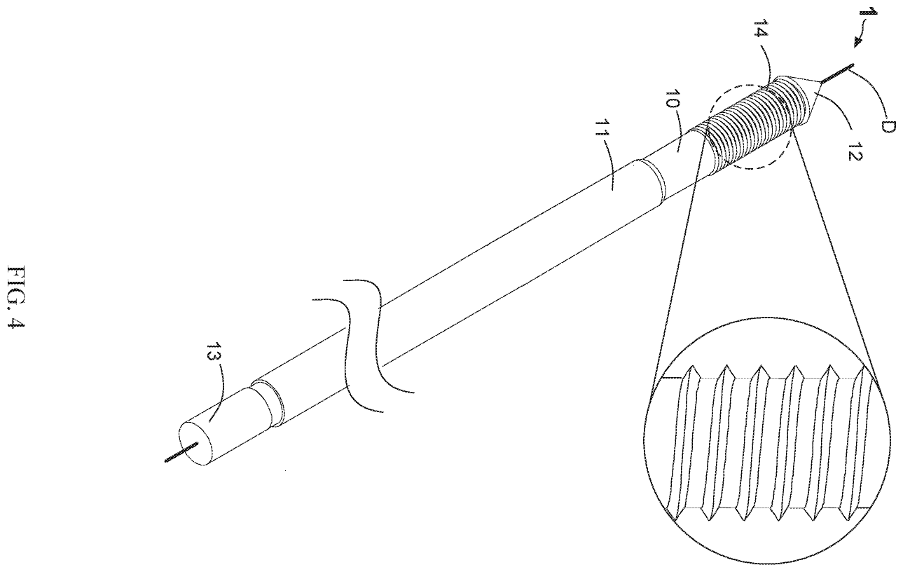

With reference to FIG. 2, there is provided a first stereo view of a catheter device according to the present invention. Moreover FIG. 3 shows an assembly framework view of the catheter structure. As FIG. 2 and FIG. 3 show, the catheter device 1 of the present invention comprises: a catheter 10, a catheter sheath 11, a tapered head 12, a guide wire D, a rotary member 13, and a helical structure 14, wherein the catheter 10 has a front-end portion 101 and a tail-end portion 102. Besides, the catheter sheath 11 is sleeved on the catheter 10, which is slidable and used for increasing the hardness of the catheter 1. Moreover, the tapered head 12 is connected to the front-end portion 101. Please simultaneously refer to FIG. 4, which illustrates a second stereo view of a catheter structure. As the FIG. 4 shows, the guide wire D is disposed on the catheter 10, and one end of the guide wire D extends out of the tapered head 12 from the internal of the catheter 10. It is worth further explaining that, the rotary member 13 is connected to the tail-end portion 102, and the other end of the guide wire D passing though the rotary member 13. On the other hand, the helical structure 14 is formed on the side of the front-end portion 101.

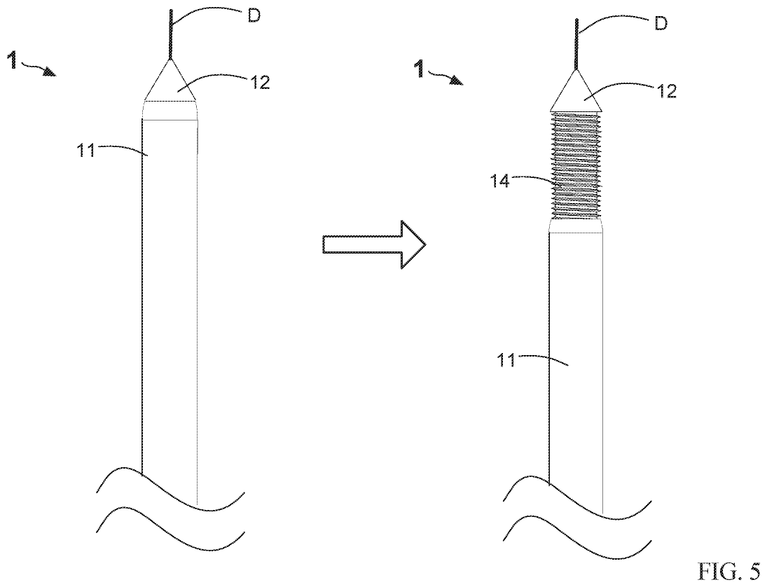

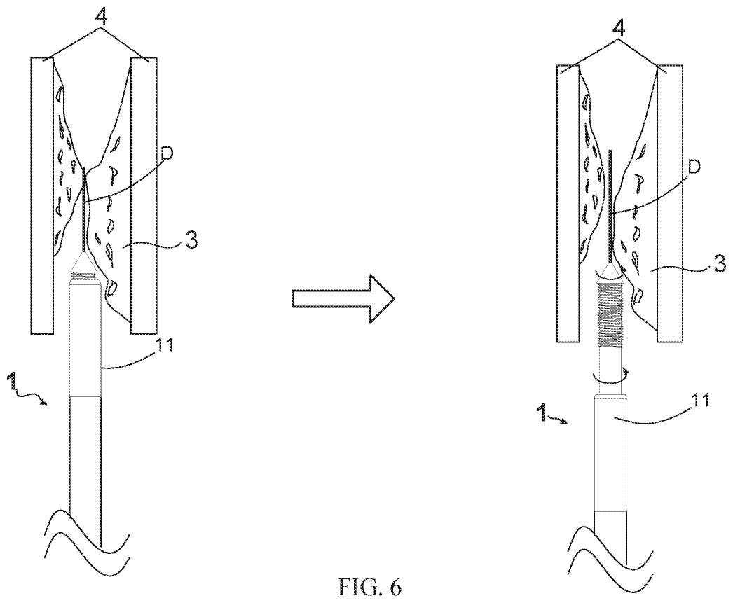

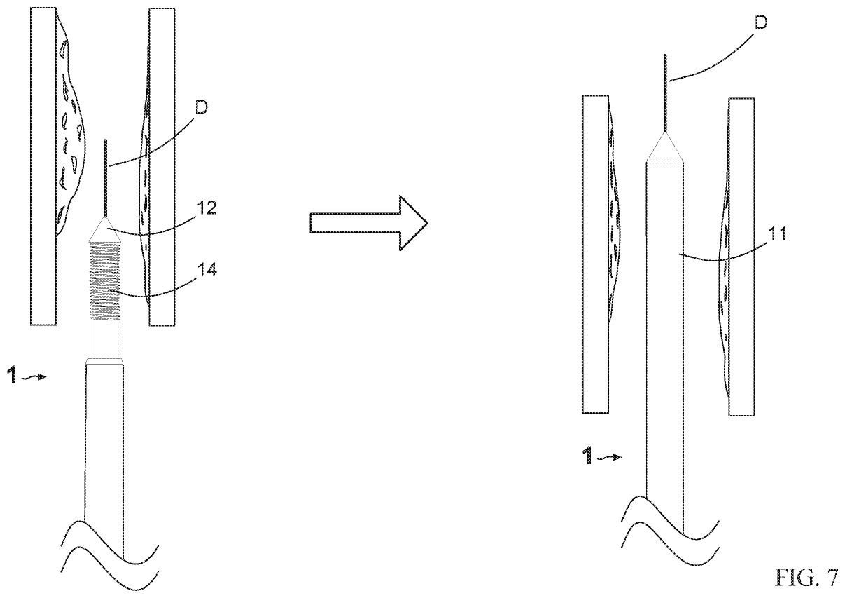

With reference to FIG. 5, which shows a first schematic view of the catheter structure. From FIG. 5, it is found that the catheter sheath 11 can slide on the front-end 101 of the catheter device 1 along a first direction, so as to cover the helical structure 14. On the contrary, by making the catheter sheath 11 slide on the front-end 101 of the catheter device 1 along a second direction contrary to the first direction, the helical structure 14 is facilitated to be revealed. Moreover, please simultaneously refer to FIG. 0.6 and FIG. 7, wherein FIG. 6 shows a second schematic view of the catheter structure, and FIG. 7 shows a third schematic view of a catheter structure. When the catheter device 1 is inserted through a blood vessel and stopped by a plaque, it is able to operate the rotary member 13 to rotate the catheter 10 and the tapered head 12; subsequently, the tapered head 12 and the helical structure 14 are used to drill the plaque of the blockage region 3, such that a blockage region 3 of the plaque for the catheter 10 is formed with a through hole therein. Therefore, the guide wire D can pass through the through hold of the blockage region 3. Consequently, the front-end 101 of the catheter 10 can pass through the blockage region 3 with the forward movement of the guide wire D. It needs to further explain that, the tapered head 12 is made of a smooth medical-grade material. In addition, the rotary member 13 is adopted to improve the operational efficiency of the rotary.

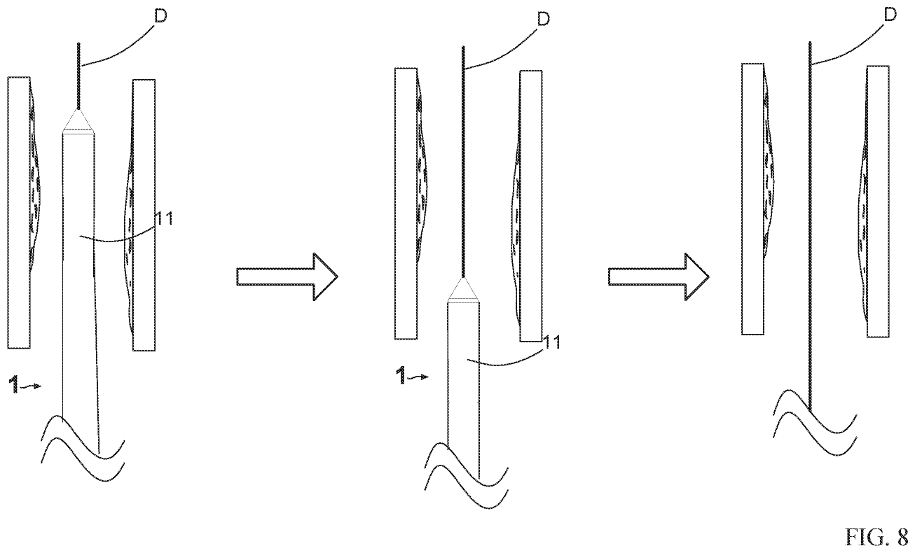

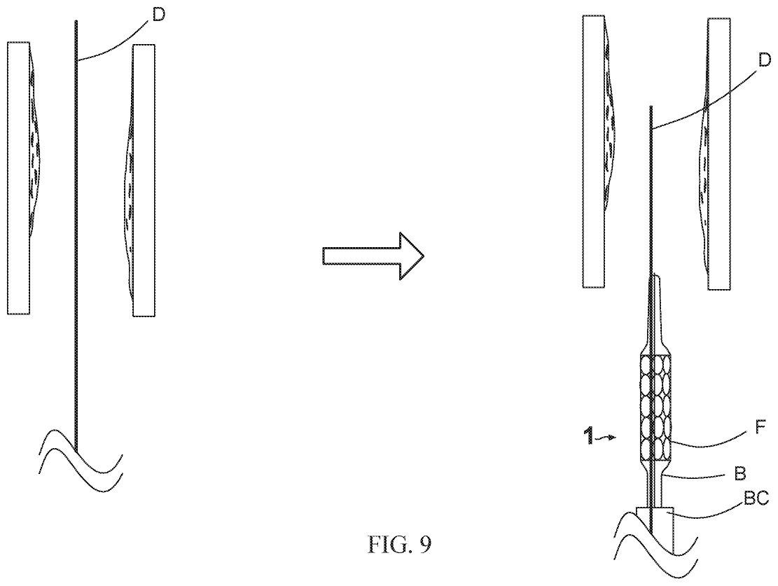

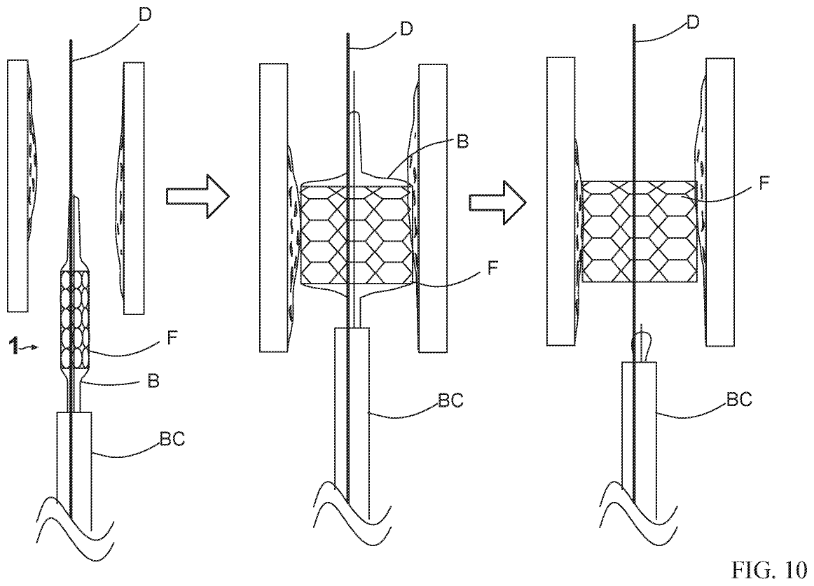

Continuously referring to FIG. 7, and please simultaneously refer to FIG. 8, FIG. 9 and FIG. 10 showing a fourth schematic view, a fifth schematic view and a sixth schematic view of the catheter structure, respectively. From FIG. 8 to FIG. 10, it is understood that, when the tapered head 12 completed to drill a through hole in the blockage region 3, the catheter 10 be removed from the blockage region 3, and the guide wire D is left behind in the blockage region 3. Afterward, a balloon catheter BC is inserted the blockage region 3 of the blood vessel with the forward movement of the guide wire D to complete a balloon angioplasty or a stent implantation.

Please refer to FIG. 4-FIG. 10 again. Herein it needs to further explain that, when the catheter device 1 is inserted through a blood vessel and stopped by a plaque of the blockage region 3. Firstly, the catheter sheath 11 sliding down along the catheter 10 to reveal the helical structure 14. Besides, the tapered head 12 can be conducted in the blockage region 3 with the forward movement of the guide wire D. Furthermore, it is able to operate the rotary member 13 to rotary the catheter 10, such that the tapered head 12 and the helical structure 14 are used to drill a through hole in the blockage region 3, and then the front-head 101 of the catheter 10 and the tapered head 12 can pass through the blockage region 3. Moreover, a catheter 10 and the tapered head 12 are removed from the blockage region 3. Subsequently, a balloon catheter BC is conducted to the blockage region 3 with the forward movement of the guide wire D. After that, the inflation component of the balloon catheter BC inflates the balloon B of the balloon catheter BC with gas or fluid, therefore the balloon filled with gas or fluid expands the stent F so as to complete a balloon angioplasty or a stent F insertion in the blockage region 3. In addition, an angiography is conducted by filling a contrast agent into the catheter 10 and then injecting the contrast agent into a blood vessel through the tapered head 12.

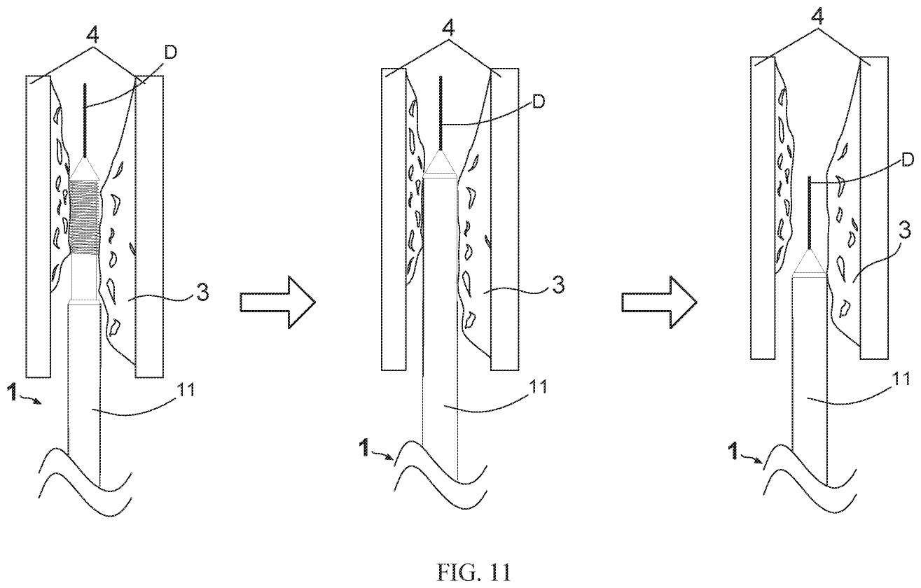

Please referring FIG. 11, which shows a seventh schematic view the catheter structure. When the catheter 10 is removed from the blockage region 3, the helical structure 14 may stuck in the plaque of the blockage region 3. At this time, by using the catheter sheath 11 covered the helical structure 14 so as to make the helical structure 14 can be removed from the blockage region 3 safely. It needs to further explain that the pipe diameter of the catheter sheath 11 is slightly bigger than the diameter of the helical structure 14.

Through above descriptions, the catheter device 1 of the present invention has been introduced completely and clearly; in summary, the present invention includes the advantages of:

(1) Conventional catheter device exhibits shortcomings of insufficient effect and long time to required. The present invention provides a catheter device 1 is able to operate the rotary member to rotary the catheter and the tapered head, such that a blockage region of the plaque for the catheter is formed with a through hole therein. Therefore, the guide wire can pass through the through hole of the blockage region, and then the front-end of the catheter can subsequently pass through the blockage region with the forward movement of the guide wire, so as to complete a balloon angioplasty or a stent implantation.

The above description is made on embodiments of the present invention. However, the embodiments are not intended to limit scope of the present invention, and all equivalent implementations or alterations within the spirit of the present invention still fall within the scope of the present invention.

What is claimed is:

1. A catheter device, comprising:
a catheter, having a front-end portion and a tail-end portion;
a catheter sheath, being sleeved on the catheter and slidable;
a tapered head, being connected to the front-end portion;
a guide wire, being disposed in the catheter, and one end of the guide wire extending out of the tapered head from the internal of the catheter;
a rotary member, being connected to the tail-end portion, and the other end of the guide wire passing though the rotary member; and
a helical structure, being formed on one side of the front-end portion.

2. The catheter device of claim 1, wherein an angiography is conducted by filling a contrast agent into the catheter and then injecting the contrast agent into a blood vessel through the tapered head.
